(19) United States
(12) Patent Application Publication
(10) Pub. No.: US 2014/0249501 A1
(43) Pub. Date: Sep. 4, 2014

(54) System and Method for Injection Protocol Management

(71) Applicant: Martin J. Uram, Pittsburgh, PA (US)

(72) Inventor: Martin J. Uram, Pittsburgh, PA (US)

(73) Assignee: MEDRAD, INC., Indianola, PA (US)

(21) Appl. No.: 13/782,062

(22) Filed: Mar. 1, 2013

(51) Int. Cl. A61M 5/172

(52) U.S. Cl. CPC A61M 5/172
USPC 604/506; 604/246

(57) ABSTRACT

A fluid injection system for controlling at least one fluid delivery system is provided. The fluid injection system includes at least one computing device in communication with the at least one fluid delivery system. The at least one computing device is configured to: display, on or with at least one display device, at least one graphical user interface configured to facilitate a user selection of at least one injection protocol of a plurality of injection protocols; receive, from at least one user, at least one selected injection protocol from the plurality of injection protocols; retrieve, from at least one remote server computer, the at least one selected injection protocol; and control the at least one fluid delivery system based at least partially on the at least one selected injection protocol.

### BACKGROUND OF THE INVENTION

1. Field of the Invention

This invention relates generally to fluid injection systems and, more specifically, to a system and method for managing fluid injection protocols and providing fluid injection systems with fluid injection protocols.

2. Background of the Invention

In injection-based therapies, various injection parameters are often required. For example, with cellular therapy fluids, specific cells have been found to require specific values for various injection parameters. These parameters are necessary for optimizing cell outputs.

Medical professionals are often in need of injection protocols for different situations and different patients. Therefore, the medical professionals need to customize a protocol for a particular situation. At the same time, other medical professionals may be in need of such customized protocols for similar situations.

Further, research and development professionals are often in need of injection protocols for different testing situations. Therefore, the research and development professionals need to customize a protocol for a particular situation.

### SUMMARY OF THE INVENTION

Generally, the present invention provides a system and method for managing injection protocols and providing injection systems with injection protocols that overcome some or all of the drawbacks and deficiencies associated with known injection systems.

According to one non-limiting embodiment of the present invention, provided is a fluid injection system for controlling at least one fluid delivery system, the fluid injection system comprising at least one computing device in communication with the at least one fluid delivery system, the at least one computing device configured to: display, on or with at least one display device, at least one graphical user interface configured to facilitate a user selection of at least one injection protocol of a plurality of injection protocols; receive, from at least one user, at least one selected injection protocol from the plurality of injection protocols; retrieve, from at least one remote server computer, the at least one selected injection protocol; and control the at least one fluid delivery system based at least partially on the at least one selected injection protocol.

According to another non-limiting embodiment, provided is an fluid injection management system, comprising: at least one injection protocol database comprising injection protocol data, the injection protocol data including a plurality of injection protocols; and at least one server computer configured to: receive a plurality of injection protocols from a plurality of client computers; store the plurality of injection protocols in the at least one injection protocol database; and transmit at least one selected injection protocol of the plurality of injection protocols to at least one medical or research facility.

According to a further non-limiting embodiment, provided is a computer-implemented method for managing a plurality of fluid injection protocols, comprising: receiving a plurality of fluid injection protocols from a plurality of user computers; storing the plurality of fluid injection protocols in at least one database; receiving, from at least one computer in communication with a fluid delivery system, a request to download at least one fluid injection protocol; and transmitting at least a portion of the at least one fluid injection protocol to the at least one computer.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

FIG. 1 is a schematic view of one embodiment of an injection protocol management system according to the principles of the present invention;

FIG. 2 is another schematic view of one embodiment of an injection protocol management system according to the principles of the present invention;

FIG. 3 is a flow diagram of a method for determining injection parameters according to the principles of the present invention;

FIG. 4 is an injection protocol according to the principles of the present invention;

FIG. 5 is an injection protocol with optimized parameters according to the principles of the present invention; and

FIG. 6 is another injection protocol according to the principles of the present invention.

### DESCRIPTION OF THE PREFERRED EMBODIMENTS

For purposes of the description hereinafter, the terms “end”, “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

As used herein, the terms “communication” and “communicate” refer to the receipt, transmission, or transfer of one or more signals, messages, commands, or other type of data. For one unit or device to be in communication with another unit or device means that the one unit or device is able to receive data from and/or transmit data to the other unit or device. A communication may use a direct or indirect connection, and may be wired and/or wireless in nature. Additionally, two units or devices may be in communication with each other even though the data transmitted may be modified, processed, routed, etc., between the first and second unit or device. For example, a first unit may be in communication with a second unit even though the first unit passively receives data, and does not actively transmit data to the second unit. As another example, a first unit may be in communication with a second unit if an intermediary unit processes data from one unit and transmits processed data to the second unit. It will be appreciated that numerous other arrangements are possible. Any known electronic communication protocols and/or algorithms may be used such as, for example, TCP/IP (including HTTP and other protocols), WLAN (including 802.11 and other radio frequency-based protocols and methods), and/or the like.

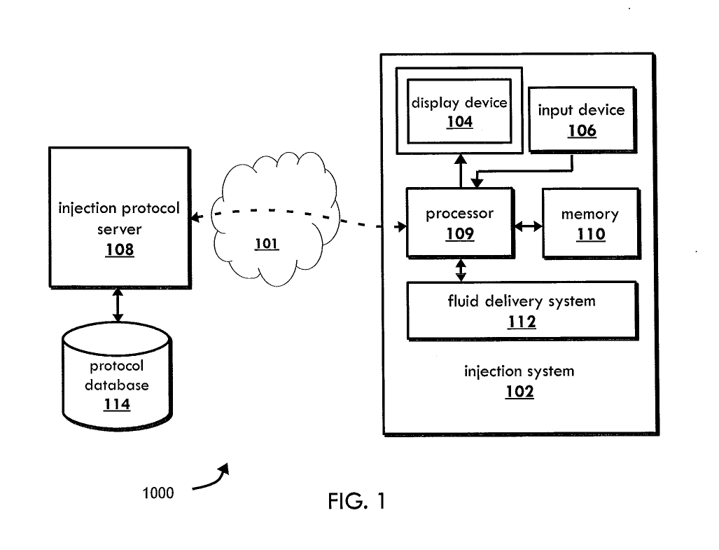

Referring to FIG. 1, an injection protocol management system 1000 is shown according to one non-limiting embodiment. The system includes an injection protocol server 108 in communication with an injection protocol database 114. The injection protocol server 108 is also in communication with an injection system 102 via a network environment 101, such as the Internet, a wide-area network (WAN), a local-area network (LAN), and/or the like. The injection system 102 is typically in a medical facility environment, such as a hospital or a medical or research laboratory, and is used for controlling and/or delivering the injection of one or more fluids to a patient, an animal, or a test receptacle (for example, a Petri dish or the like). In the illustrated example, the injection system 102 includes a display device 104, an input device 106, a processor 109, a memory unit 110, and a fluid delivery system 112.

In some non-limiting embodiments, the processor 109, memory 110, input device 106, and/or the display device 104 of the injection system 102 may be part of a separate computer that is in communication with the fluid delivery system 112. In other non-limiting embodiments, the processor 109, memory 110, input device 106, and/or the display device 104 may be part of or integrated with a housing of the fluid delivery system 112. In further non-limiting embodiments, the injection system 102 may include a combination of one or more separate computers in communication with the fluid delivery system 112 and internal components (e.g., processors, memory, etc.) of the fluid delivery system 112. It will be appreciated that various other components and/or modules may be included with the injection system 102 and/or fluid delivery system 112. Further, numerous other arrangements are possible and various components may be integrated with and/or separated from one another.

With continued reference to FIG. 1, a user of the injection system 102 is provided, through the display device 104, with various options and/or interfaces for searching and/or selecting one or more injection protocols for a particular patient or treatment. Using the input device 106, which may include a keyboard, mouse, touchscreen, smart phone, tablet, phablet, voice recognition software, and/or the like, a user is able to search for and/or select an injection protocol from the injection protocol database 114. The interfaces may be local to the injection system 102 and populated with data received from the injection protocol server 108, or may be provided by a server such as, but not limited to, the injection protocol server 108. After selecting one or more injection protocols that are stored in the injection protocol database 114, the user is able to download, to the memory 110 of the injection system 102, the one or more desired protocols. The protocols stored in memory 110 may then be communicated to the fluid delivery system 112 and used to control the injection process for one or more patients.

Still referring to FIG. 1, the fluid delivery system 112, in one non-limiting embodiment, may be physically separate from and in communication with other components of the injection system 102, such as the processor 109, memory unit 110, display device 104, input device 106, and/or the like. In another example, the fluid delivery system 112 may include such components, and some or all components may be incorporated into a housing. Thus, the system 1000 may be implemented with various types of injection systems and fluid delivery systems, including injection and fluid delivery systems that are manually configured and/or programmed, and systems that are automatically configured and/or programmed based on injection protocol data and/or commands.

The term “injection system,” as used herein, refers to one or more hardware and/or software components, systems, modules, and/or the like used to deliver fluid to a patient, an animal, or a test receptacle. It will be appreciated that the various components and/or modules of the injection system may be included in a single housing, may be separated, and/or may be arranged in any other operable way. The term “fluid delivery system,” as used herein, refers to a portion of an injection system 102, or separate components or devices in communication with an injection system 102, which deliver fluid to patients, animals, or test receptacles, and/or control fluid delivery. In some non-limiting embodiments, the fluid delivery system 112 may include a syringe motor, actuator, interface, one or more pumps, one or more pump cartridges, one or more ports for receiving pump cartridges, tubing or other fluid paths, and/or the like. Examples of injection and fluid delivery systems that can be used with the injection protocol management system 1000 include, but are not limited to, those described in U.S. Patent Application Publication No. 2012/0123257 to Stokes et al., U.S. Patent Application Publication No. 2008/0086087 to Spohn et al., and WIPO Patent Application Publication No. WO/2012/155035 to Shearer et al., all of which are hereby incorporated by reference in their entirety. The fluid delivery systems may include various components such as, but not limited to, those discussed in U.S. Pat. No. 8,337,456 to Schriver et al. and U.S. Pat. No. 8,147,464 to Spohn et al., which are also hereby incorporated by reference in their entirety.

As used herein, the terms “injection protocol database” and “protocol database” refer to one or more data structures configured to store injection protocol data. In some non-limiting embodiments, the injection protocol database 114 may include data structured as one or more tables, trees, arrays, objects, and/or other like data structures. The data may be arranged by pharmaceutical, cell type, identifier, therapeutic use, protocol name, and/or the like. It will be appreciated that, with some forms of structured data, the data may be arranged in any number of ways depending on how it is queried. In non-limiting embodiments utilizing an object-oriented database, the protocols themselves may be individual objects with various attributes and parameters. It will be appreciated that any other data storage methods and/or data structures may be used.

In one non-limiting embodiment, injection protocol data may include one or more parameters and/or variables such as, but not limited to, a flow rate, a flow acceleration and/or deceleration rate, a flow pressure, a number of phases, various identifiers for drugs, contrast fluids, cells, injection systems, containers, tubing and/or other fluid delivery system components, an initial cell concentration, an initial pharmaceutical concentration, a timing or sequence for injection steps, a particular use, a variable associated with a fluid containing cells and/or pharmaceuticals, a variable associated with a fluid path for the fluid delivery system, a total fluid volume, and/or other like parameters. In the injection protocol database, sets of one or more parameters may be associated with one or more pharmaceuticals, cell types, injection types, protocol names or identifiers, therapeutic uses, target organ or body area for the injected fluid, and/or the like.

In one non-limiting embodiment, the injection protocol management system 1000 is used for delivering cellular therapies to patients. In this example, the injection protocols include one or more parameters associated with one or more cells to optimize cell outputs (e.g., apoptosis, differentiation, proliferation, and/or the like). Medical professionals, researchers, and other individuals or entities may experiment with various injection parameters for different cells and/or therapies. This testing data, or results derived from such testing data, may be used to generate the injection parameters for a protocol.

In one non-limiting embodiment, the injection protocols may be generated, at least in part, based on the injection protocol data and patient-based factors. The patient-based factors may include various patient parameters including, but not limited to, a patient age, weight, body mass index (BMI), gender, medical history, and/or the like. Thus, injection protocols may factor in such patient parameters by weighing, multiplying, or otherwise calculating injection parameters based on the patient parameters. It will be appreciated that such factoring and/or calculations may be performed by the injection protocol server 108, the injection system 102, or any combination of computing devices local and/or remote to the injection system 102.

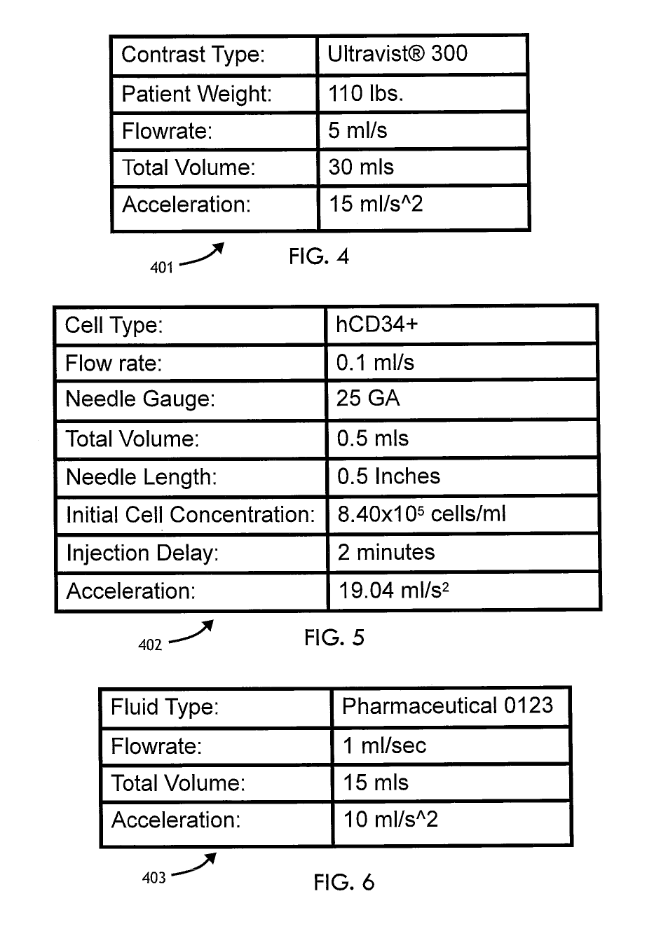

Referring now to FIG. 4, shown is a data structure 401 for an example injection protocol according to one non-limiting embodiment. As can be seen, the injection protocol specifies contrast type, patient weight, flow rate, total volume, and acceleration. In this example, a patient weighing 110 lbs will be provided with 30 milliliters of Ultravist® 300 contrast fluid at a flow rate of 5 ml/sec and an acceleration of 15 ml/sec². Referring to FIG. 6, shown is a data structure 403 for another example injection protocol according to a non-limiting embodiment. The data structure 403 representing the injection protocol shown in FIG. 6 is for a pharmaceutical fluid and has a flow rate of 1 ml/sec, a total volume of 15 milliliters, and an injection acceleration of 10 ml/sec². Although the injection protocol data structures 401, 403 are shown in a tabular format, it will be appreciated that any number of data structures may be used such as, for example, tabular and object-oriented databases, trees, arrays, and/or the like.

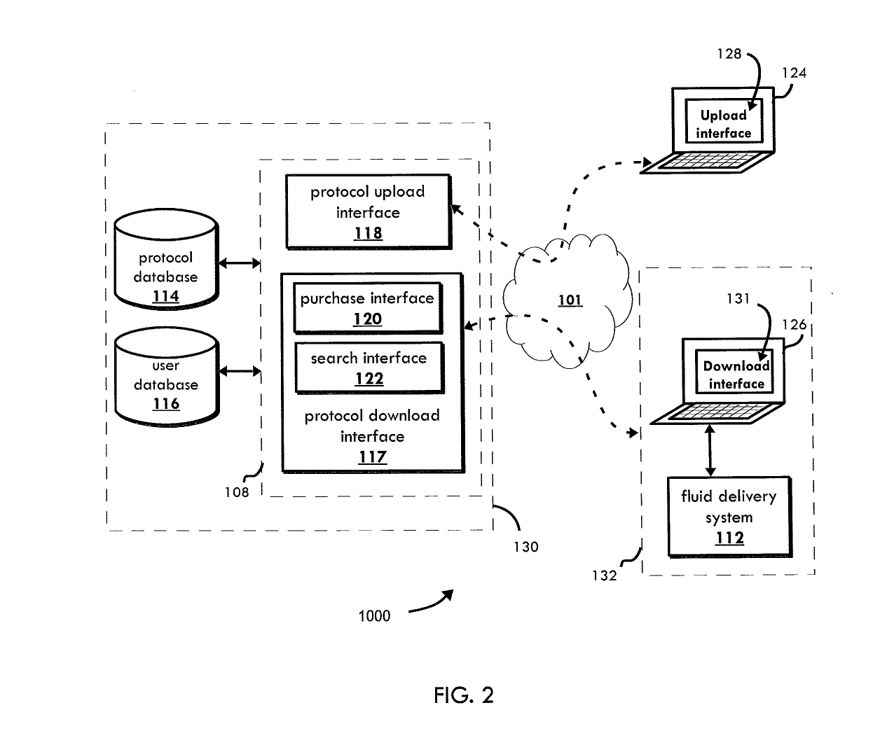

Referring now to FIG. 2, a further non-limiting embodiment of the injection protocol management system 1000 is shown. An injection protocol host 130 includes an injection protocol server 108 in communication with an injection protocol database 114 and a user database 116. The injection protocol server 108 includes a protocol download interface 117 and a protocol upload interface 118. In one non-limiting embodiment, the injection protocol server 108 is a web server and the protocol download interface 117 and the protocol upload interface 118 are different portals, interfaces, and/or web pages made available by the web server.

The interfaces 117, 118 may be integrated or separate subsystems, such as modules, components, and/or the like, that allow different graphical interfaces, portals, web pages, and/or the like to be accessed by and/or provided to different remote users. In some embodiments, the interfaces 117, 118 may be two separate web pages hosted by the server 108. The protocol upload interface 118 may transmit, to a provider computer 124, display data configured to generate an upload graphical user interface (GUI) 128 to enable a protocol provider, such as a physician or other type of medical or research professional, to upload injection protocols to the server 108. The protocol upload interface 118 may therefore include payment history and/or activity history information relating to third-party downloads, accruement of royalties or payments for third-party downloads, and/or other like information relating to the protocols uploaded by the user. The upload GUI 128 may also include input fields, selection options (i.e., radio buttons, check boxes, etc.), and buttons to facilitate user input.

In one non-limiting embodiment, users that upload protocols to the injection protocol server 108 are associated with a provider account that allows the users to login and monitor the download and/or purchase activity associated with the protocols that they uploaded. For example, payment history information may be generated based at least in part on a number of downloads, and/or an aggregate revenue generated by such downloads, of protocols that the user uploaded and/or otherwise provided. It will be appreciated that various payment arrangements are possible to reward and/or compensate the protocol providers.

With continued reference to FIG. 2, the protocol download interface 117 may be accessed by users at medical or research facilities 132 seeking an injection protocol to be used with a fluid delivery system 112. The medical or research facility 132 may include a medical or research facility computer 126 and a fluid delivery system 112. As already explained, in embodiments, the medical or research facility computer 126 may be incorporated with and/or separate from the fluid delivery system 112, or an overall injection system 102 (not shown) that includes the fluid delivery system 112. In one example, the medical or research facility computer 126 may be in communication with the fluid delivery system 112, and configured to transmit commands, protocols, and other types of data to the fluid delivery system 112 to enable operation. In another example, a user may access the host 130 with the medical or research facility computer 126 to obtain one or more protocols, and may manually program the fluid delivery system 112 and/or the injection system 102 (not shown) according to the retrieved protocols.

Still referring to FIG. 2, the medical or research facility computer 126 may display a download GUI 131 based on display data received from the injection protocol server 108. The download GUI 131 may include, for example, one or more web pages. A purchase interface 120 may generate and/or display various graphical interfaces to accept payment from users in exchange for downloading one or more injection protocols. For example, the purchase interface 120 may process credit card transactions, communicate with a third-party credit card processor, or withdraw from a user's account balance based on the protocols downloaded or requested by the user. A search interface 122 may generate and/or display various GUIs on the medical or research facility computer 126 to allow users to search the injection protocol database 114 by one or more search parameters. The search interface 122 may, for example, facilitate a user to construct one or more queries to retrieve information from the injection protocol database 114. Searches may be conducted based on any parameter or variable used to define or organize the protocols in the injection protocol database 114 such as, but not limited to, various identifiers for drugs or pharmaceuticals, contrast fluids, cells, injection systems, patient conditions, and/or the like.

With continued reference to FIG. 2, in one non-limiting embodiment, the user database 116 may be in communication with the injection protocol server 108 and may be used to store user data, credential data, and/or the like. The user data and/or credential data may be used to authenticate users, and may include a user name, login, password, location information, medical or research facility information, injection system information, and/or the like. In embodiments, the user database 116 may also be used to manage user data for protocol providers that upload injection protocols to the protocol database 114.

In one non-limiting embodiment, injection protocols stored in the protocol database 114 may be ranked in various ways. Protocol rankings may include, for example, a user-specified ranking, a number of times downloaded and/or sold, a number of times viewed, a date first uploaded, and/or the like. User rankings may be based on a grading or number system (e.g., a ranking Y out of X number of units, where Y is equal or less than X). Rankings may also be used for each protocol, or various aspects of each protocol (e.g., efficiency, effectiveness, cost, and/or the like). Moreover, users may comment or add notes to various protocols to provide information or advice on using the protocols, to review or evaluate the protocols, or to otherwise provide information relevant to one or more protocols. Media content, such as photographs, pictures, video data, audio data, and/or the like, may also be uploaded by users to be associated with one or more protocols. Such media content may allow a user to evaluate whether a particular protocol would be appropriate for their use.

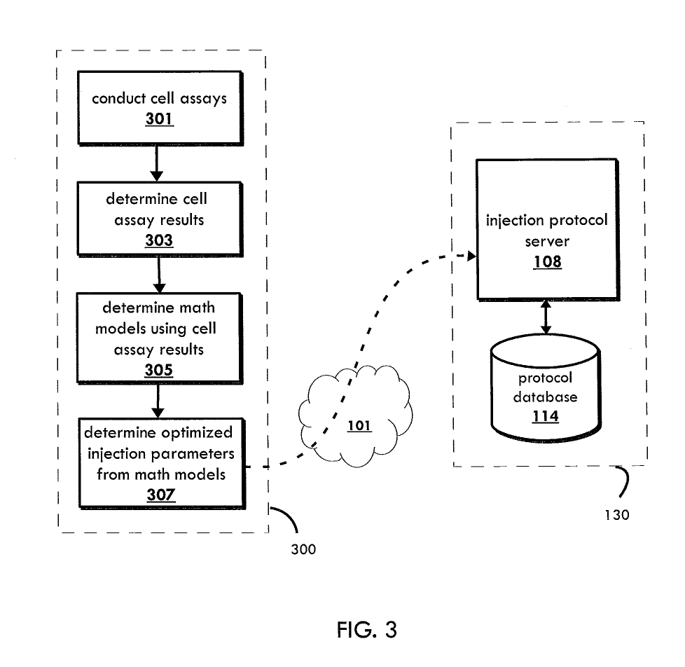

Referring to FIG. 3, a system and method for determining injection parameters with regard to cells is shown according to one non-limiting embodiment. Exemplary parameter generation methods, and associated methods for assaying various types of cells, are explained in more detail in International Application No. PCT/US2012/066792, titled “Systems And Methods For Injecting Cellular Fluids,” which is hereby incorporated by reference in its entirety. The parameter determination method 300 begins with a first step 301 in which cell assays are conducted. During this step, various aspects of cells, such as cytotoxicity, rates or intervals associated with cell death, and/or the like, may be assayed with various techniques known by those skilled in the art. During a next step 303, the results of the cell assays performed may be determined by manual or computational analysis. In step 305, math models are determined based on the results determined in step 303. At a final step 307, the optimized injection parameters may be determined from the math models determined in step 305. The optimized injection parameters may then be uploaded or otherwise transmitted to the injection protocol server 108 and/or the injection protocol database 114. Likewise, the optimized injection parameters may be downloaded and used by others.

Referring now to FIG. 5, an example of optimized parameters used in a cell injection protocol is shown according to one non-limiting embodiment. An optimized injection parameter data structure 402 is shown in a tabular format and includes a cell type, a flow rate, a needle gauge, a total volume, a needle length, an initial cell concentration, an injection delay, and an acceleration. In this example, the cell type is hCD34+, the flow rate is 0.1 ml/sec, the needle gauge is 25 GA, the total volume is 0.5 mls, the needle length is 0.5 inches, the initial cell concentration is 8.40×10⁵ cells/ml, the injection delay is 2 minutes, and the acceleration is 19.04 ml/s². Although the optimized injection parameter data structure 402 is shown in a tabular format, it will be appreciated that any number of data structures may be used such as, for example, tabular and object-oriented databases, arrays, trees, and/or the like.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

What is claimed is:

1. A fluid injection system for controlling at least one fluid delivery system, the fluid injection system comprising at least one computing device in communication with the at least one fluid delivery system, the at least one computing device configured to:
display, on or with at least one display device, at least one graphical user interface configured to facilitate a user selection of at least one injection protocol of a plurality of injection protocols;
receive, from at least one user, at least one selected injection protocol from the plurality of injection protocols;
retrieve, from at least one remote server computer, the at least one selected injection protocol; and
control the at least one fluid delivery system based at least partially on the at least one selected injection protocol.

2. The fluid injection system of claim 1, wherein the at least one computing device is further configured to transmit, to the at least one remote server computer, a request to search at least one injection protocol database based at least partially on at least one search parameter, the at least one search parameter comprising at least one of the following: a cellular treatment, a target organ or body area for the injected fluid, a medication, a condition, or any combination thereof.

3. The fluid injection system of claim 2, wherein the at least one computing device is further configured to receive, from the at least one remote server computer, data configured to generate at least one search interface configured to search the at least one injection protocol database.

4. The fluid injection system of claim 1, wherein the at least one computing device is further configured to receive payment information from the at least one user, and transmit at least a portion of the payment information to the at least one remote server computer.

5. The fluid injection system of claim 1, wherein the at least one computing device is further configured to display payment history data, the payment history data generated based at least partially on a number of downloads of the plurality of injection protocols.

6. The fluid injection system of claim 1, wherein the at least one computing device is further configured to:
receive, from the at least one user, at least one patient parameter; and
generate at least one injection parameter based at least partially on the at least one patient parameter and the at least one selected injection protocol.

7. The fluid injection system of claim 6, wherein the at least one patient parameter comprises at least one of a patient weight, a patient age, a patient gender, a patient medical history, or any combination thereof.

8. A fluid injection management system, comprising:
at least one injection protocol database comprising injection protocol data, the injection protocol data including a plurality of injection protocols; and
at least one server computer configured to:
receive a plurality of injection protocols from a plurality of client computers;
store the plurality of injection protocols in the at least one injection protocol database; and
transmit at least one selected injection protocol of the plurality of injection protocols to at least one medical or research facility.

9. The fluid injection management system of claim 8, wherein the at least one injection protocol comprises at least one of the following: a flow rate, a flow acceleration, a flow deceleration, a flow pressure, a number of phases, a tube identifier, a fluid delivery system component identifier, an initial cell concentration, a drug or pharmaceutical identifier, an initial pharmaceutical concentration, a total fluid volume, a timing, or any combination thereof.

10. The fluid injection management system of claim 8, wherein the at least one server computer is further configured to receive a request to search the at least one injection protocol database based at least partially on at least one search parameter, the at least one search parameter comprising at least one of the following: a cellular treatment, a target organ or body area for the injected fluid, a medication, a condition, or any combination thereof.

11. The fluid injection management system of claim 10, wherein the at least one server computer is further configured to transmit, to the at least one medical or research facility, at least one search interface configured to search the at least one injection protocol database.

12. The fluid injection management system of claim 8, wherein the at least one server computer is further configured to receive payment information from the at least one medical or research facility.

13. The fluid injection management system of claim 8, wherein the at least one server computer is further configured to transmit, to the at least one client computer, payment history data, the payment history data generated based at least partially on a number of downloads of the at least one injection protocol.

14. The fluid injection management system of claim 8, wherein the at least one medical or research facility comprises at least one injection system configured to facilitate a user selection of the at least one selected injection protocol from the plurality of injection protocols.

15. The fluid injection management system of claim 8, wherein the at least one server computer is further configured to receive, from the at least one medical or research facility, at least one patient parameter, wherein the at least injection protocol is generated based at least partially on the at least one patient parameter.

16. The fluid injection management system of claim 8, wherein the at least one server computer is further configured to determine rankings for at least a portion of the plurality of injection protocols, the rankings determined based at least partially on at least one of the following: a ranking received from a user, a number of times downloaded, a number of times used, a number of comments, or any combination thereof.

17. A computer-implemented method for managing a plurality of fluid injection protocols, comprising:
receiving a plurality of fluid injection protocols from a plurality of user computers;
storing the plurality of fluid injection protocols in at least one database;
receiving, from at least one computer in communication with a fluid delivery system, a request to download at least one fluid injection protocol; and
transmitting at least a portion of the at least one fluid injection protocol to the at least one computer.

18. The computer-implemented method of claim 17, wherein each of the plurality of fluid injection protocols comprise at least one of the following: a flow rate, a flow acceleration, a flow deceleration, a tube identifier, a fluid delivery system component identifier, a drug or pharmaceutical identifier, an initial cell concentration, a total fluid volume, an initial pharmaceutical concentration, an injection timing, or any combination thereof.

19. The computer-implemented method of claim 17, further comprising receiving a request to search the at least one database based at least partially on at least one search parameter, the at least one search parameter comprising at least one of the following: a cellular treatment, a target organ or body area for the injected fluid, a medication, a condition, or any combination thereof.

20. The computer-implemented method of claim 17, further comprising transmitting, to the at least one computer, at least one search interface configured to search the at least one database.

21. The computer-implemented method of claim 17, further comprising receiving, from the at least one computer, payment information for purchasing the at least one fluid injection protocol.

22. The computer-implemented method of claim 17, further comprising transmitting, to at least one protocol provider computer, payment history data, the payment history data generated based at least partially on a number of downloads of at least one fluid injection protocol uploaded by the at least one protocol provider.

23. The computer-implemented method of claim 17, further comprising receiving, from the at least one computer, at least one patient parameter, wherein the at least one fluid injection protocol is generated based at least partially on the at least one patient parameter.

24. The computer-implemented method of claim 17, further comprising determining rankings for at least a portion of the plurality of fluid injection protocols, the rankings determined based at least partially on at least one of the following: a ranking received from a user, a number of times downloaded, a number of times used, a number of comments, or any combination thereof.
